(19) United States
(12) Patent Application Publication
(10) Pub. No.: US 2025/0032704 A1
(43) Pub. Date: Jan. 30, 2025

(54) MEMS MICROPUMP WITH MULTI-CHAMBER CAVITY FOR A DEVICE FOR DELIVERING INSULIN

(71) Applicant: AITA BIO INC., New York, NY (US)

(72) Inventors: Dilan Casanovas Mack, New York, NY (US); Jacob Menture, New York, NY (US); Laurence Heads, Frisco, TX (US)

(21) Appl. No.: 18/711,760

(22) PCT Filed: Jan. 16, 2023
(86) PCT No.: PCT/US23/10858

§ 371 Date: May 20, 2024

Related U.S. Application Data
(60) Provisional application No. 63/300,885, filed on Jan. 19, 2022.

(51) Int. Cl. A61M 5/145; A61M 5/142; A61M 5/158; A61M 5/168

(52) U.S. Cl. CPC A61M 5/14586; A61M 5/14224; A61M 5/158; A61M 5/168; A61M 2202/0007; A61M 2202/04; A61M 2205/0216; A61M 2205/0244; A61M 2205/0294; A61M 2205/3303; A61M 2205/3327; A61M 2205/3331; A61M 2205/50; A61M 2230/005; A61M 2230/201

(57) ABSTRACT

A MEMS device is disclosed for a device for delivering insulin into a subcutaneous tissue of a user. The device for delivering insulin is configured to be mounted to the user, the MEMS device including an inlet port and outlet port, the MEMS device configured as a micropump to pump the insulin from the inlet port to the outlet port, the MEMS device comprising: first and second wafers that define a cavity therebetween that communicates with the inlet and outlet ports, thereby creating a fluid path for a flow of the insulin from the inlet port to the outlet port, the first wafer configured as a membrane, the cavity comprises: a first chamber configured as a pumping chamber of the micropump; and a second chamber and a third chamber configured as valve chambers of the micropump, wherein the second chamber communicates with the first chamber and the inlet port and the third chamber communicates with the first chamber and the outlet port and wherein the first, second chamber and third chamber form the fluid path through the micropump from the inlet port to the outlet port.

### CROSS-REFERENCE TO RELATED APPLICATIONS

This application claims priority to U.S. provisional application No. 63/300,885, filed Jan. 19, 2022 entitled “MEMS Micropump With Three Chamber Cavity,” which is incorporated by reference herein.

### FIELD OF THE INVENTION

The present invention relates to a MEMS micropump with a multi-chamber cavity for a device for delivering insulin.

### BACKGROUND OF THE INVENTION

Insulin delivery devices help people with diabetes to conveniently manage their blood sugar. These devices deliver insulin at specific times. Insulin patch pumps or pods are one type of insulin pump. The pods are wearable devices that adhere to the skin of a user using an adhesive patch. The pods incorporate a pump for delivering insulin from through a chamber and internal cannula based on separately acquired CGM sensor readings. The pumps typically have a mechanical architecture and rely on bulky components such as gears and stepper motors to deliver the drug into a patient. The device is controlled wirelessly by a handheld controller.

It would be advantageous to provide improvements to insulin pumps described above.

### SUMMARY OF THE INVENTION

The MEMS micropump is disclosed with a multi-chamber cavity for a device for delivering insulin.

In accordance with an embodiment of the present disclosure, a MEMS device for a device for delivering medicament into a subcutaneous tissue of a user, the device for delivering medicament configured to be mounted to the user, the MEMS device including an inlet port and outlet port, the MEMS device configured as a micropump to pump the medicament from the inlet port to the outlet port, the MEMS device comprising: first and second wafers that define a cavity therebetween that communicates with the inlet and outlet ports, thereby creating a fluid path for a flow of the medicament from the inlet port to the outlet port, the first wafer configured as a membrane, the cavity comprises: a first chamber configured as a pumping chamber of the micropump; and a second chamber and a third chamber configured as valve chambers of the micropump, wherein the second chamber communicates with the first chamber and the inlet port and the third chamber communicates with the first chamber and the outlet port and wherein the first, second chamber and third chamber form the fluid path through the micropump from the inlet port to the outlet port, wherein the first wafer as a membrane is configured to deflect creating a pressure difference within the cavity and thereby draw medicament from the inlet port into the first chamber or displace medicament from the first chamber toward the outlet port.

In accordance with another embodiment of the present disclosure, a device for delivering insulin to a user for diabetes management, the device configured to be mounted the user, the device comprising: an infusion catheter for infusing the insulin into a subcutaneous tissue of the user; a MEMS device configured as a micropump for pumping the insulin through the micropump, the MEMS device in fluid communication with the infusion catheter, the MEMS device comprising: an inlet port for receiving the insulin and outlet port for releasing the insulin to supply; first and second wafers that define a cavity that communicates with the inlet and outlet ports, thereby creating a fluid path for a flow of the insulin from the inlet port to the outlet port, the cavity including a first chamber configured as a pump chamber and/or a valve chamber of the micropump and a second chamber configured as a pump chamber and/or a valve chamber, the first chamber and second chamber in communication therewith creating the fluid path between the inlet and outlet ports; a first piezoelectric actuator layered on the first wafer and configured to deform the first wafer relative to the first chamber; a second piezoelectric actuator layered on the first wafer and configured to deform the first wafer relative to the second chamber, wherein the first wafer as a membrane is configured, upon deformation, to create a pressure difference within the cavity and thereby draw insulin from the inlet port into the first chamber and/or second chamber or displace fluid from the first chamber and/or second chamber toward the outlet port.

In accordance with another embodiment of the disclosure, a device for delivering medicament to a user, the device configured to be mounted the user, the device comprising: a sensor for sensing a biomarker in the user; an infusion catheter for infusing the medicament into tissue of the user based on the biomarker sensed by the sensor; a MEMS device configured as a micropump for pumping the medicament through the micropump, the MEMS device in fluid communication with the infusion catheter, the MEMS device comprising: an inlet port for receiving the medicament and outlet port for releasing the medicament to supply; first and second wafers that define a cavity that communicates with the inlet and outlet ports, thereby creating a fluid path for a flow of the medicament from the inlet port to the outlet port, the cavity including a first chamber configured as a pump chamber, a second chamber configured as a valve chamber and a third chamber configured as a valve chamber, the first chamber in communication with the second chamber and third chamber therewith creating the fluid path between the inlet and outlet ports; a first piezoelectric actuator layered on the first wafer and configured to deform the first wafer relative to the first chamber; a second piezoelectric actuator layered on the first wafer and configured to deform the first wafer relative to the second chamber; and a third piezoelectric actuator layered on the first wafer and configured to deform the first wafer relative to the third chamber, wherein the first wafer as a membrane is configured, upon deformation, to create a pressure difference within the cavity and thereby draw medicament from the inlet port into the second chamber or displace medicament from the first chamber toward the outlet port.

In accordance with another embodiment of the disclosure, a device for delivering insulin to a user for diabetes management, the device configured to be mounted the user, the device comprising: an infusion catheter for infusing the insulin into a subcutaneous tissue of the user; a MEMS device configured as a micropump for pumping the insulin through the micropump, the MEMS device in fluid communication with the infusion catheter, the MEMS device comprising: an inlet port for receiving the insulin and outlet port for releasing the insulin to supply; first and second wafers that define a cavity that communicates with the inlet and outlet ports, thereby creating a fluid path for a flow of the insulin from the inlet port to the outlet port, the cavity including a first chamber configured as a pump chamber, a second chamber configured as a valve chamber and a third chamber configured as a valve chamber, the first chamber in communication with the second chamber and third chamber therewith creating the fluid path between the inlet and outlet ports; a first piezoelectric actuator layered on the first wafer and configured to deform the first wafer relative to the first chamber; a second piezoelectric actuator layered on the first wafer and configured to deform the first wafer relative to the second chamber; and a third piezoelectric actuator layered on the first wafer and configured to deform the first wafer relative to the third chamber, wherein the first wafer as a membrane is configured, upon deformation, to create a pressure difference within the cavity and thereby draw insulin from the inlet port into the second chamber or displace insulin from the first chamber toward the outlet port.

In accordance with another embodiment of the disclosure, a device for delivering insulin to a user for diabetes management, the device configured to be mounted the user, the device comprising: an infusion catheter for infusing the insulin into a subcutaneous tissue of the user; a MEMS device configured as a micropump for pumping the insulin through the micropump, the MEMS device in fluid communication with the infusion catheter, the MEMS device comprising: an inlet port for receiving the insulin and outlet port for releasing the insulin to supply; first and second wafers that define a cavity that communicates with the inlet and outlet ports, thereby creating a fluid path for a flow of the insulin from the inlet port to the outlet port, the cavity including a first chamber configured as a pump chamber and as a circular section, a second chamber configured as a valve chamber and as a circular section, a third chamber configured as a valve chamber and as a circular section, a first channel between the first chamber and second chamber and a second channel between the first chamber and third chamber, the first and second channels thereby enabling communication within the cavity and the flow of insulin between the inlet and outlet ports; a first piezoelectric actuator layered on the first wafer and configured to deform the first wafer relative to the first chamber; a second piezoelectric actuator layered on the first wafer and configured to deform the first wafer relative to the second chamber; and a third piezoelectric actuator layered on the first wafer and configured to deform the first wafer relative to the third chamber, wherein the first wafer as a membrane is configured, upon deformation, to create a pressure difference within the cavity and thereby draw insulin from the inlet port into the second chamber or displace insulin from the first chamber toward the outlet port.

### BRIEF DESCRIPTION OF DRAWINGS

FIG. 1 depicts a perspective exploded view of an example micropump for a device for delivering insulin to a user.

FIG. 2 depicts a cross sectional view of the micropump in FIG. 1 in fully formed configuration.

FIG. 3 depicts a perspective exploded view of another example micropump for a device for delivering insulin to a user.

FIGS. 4 and 5 depict a cross-sectional perspective view and top view of a valve section of the micropump in FIG. 1 including a valve lip.

FIG. 6 depicts a block diagram of example components of an example device for delivering insulin.

FIG. 7 depicts a perspective exploded view of another example micropump for a device for delivering insulin to a user

### DETAILED DESCRIPTION OF THE INVENTION

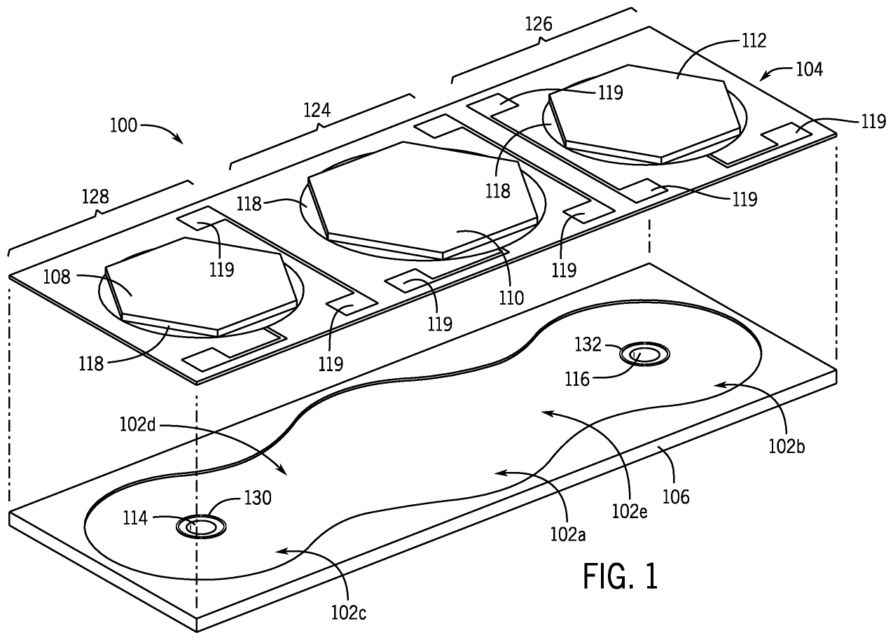

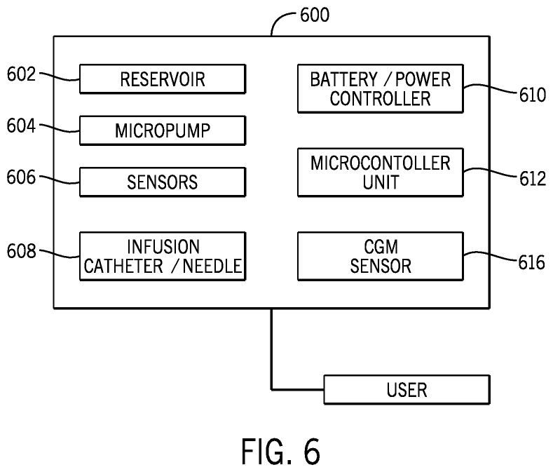

FIG. 1 depicts a perspective exploded view of an example micropump (or pump) 100 for a device for delivering insulin to a user (described in more detail below). That is, micropump 100 is part (component) of the device for delivering insulin (also referred to as a delivery device) that is configured as a wearable apparatus that is mounted on the user via an adhesive. The device is a component of an infusion system for diabetes management in which continuous glucose monitoring (CGM), insulin delivery and control functionality are provided to ensure insulin is delivered at very precise rates. In short, the device (600 in FIG. 6) includes several components or modules including, among other components, a reservoir for storing the insulin or other medicament, control circuitry (integrated circuit—IC), battery for powering the IC, an infusion catheter or needle and a continuous glucose monitoring (CGM) sensor or other sensor for sensing other biomarkers. This is described in more detail below with respect to FIG. 6.

Micropump 100 is a MEMS (micro-electro-mechanical systems) device, as known to those skilled in the art, that can be used for pumping fluid, valves used for regulating flow, actuators used for moving or controlling the micropump and valves and/or sensors used for sensing pressure and/or flow. The MEMS device incorporates one or more piezoelectric elements or devices (also known herein as piezoelectric transducers), as known to those skilled in the art. Example piezoelectric devices include piezoelectric actuators and various types of MEMS sensors. As described in more detail below, the piezoelectric devices function as the active element(s) of a pump for pumping fluid and valves for preventing fluid flow and/or a sensor for sensing pressure or flow. (However, various types of MEMS sensors can be used as the sensing elements of the architecture.) Further, other MEMS or non-MEMS structures or technology may also be used to achieve desired results as known to those skilled in the art.) Micropump 100 may be used in the drug infusion system as identified above for diabetes management including infusing a drug (i.e., medication) or other fluid to a patient (user). Medication may include small molecule pharmaceutical solutions, large molecule or protein drug solutions, saline solutions, blood or other fluids known to those skilled in the art. Insulin is an example fluid and described below with respect to micropump 100. However, micropump 100 may be used in other environments known to those skilled in the art.

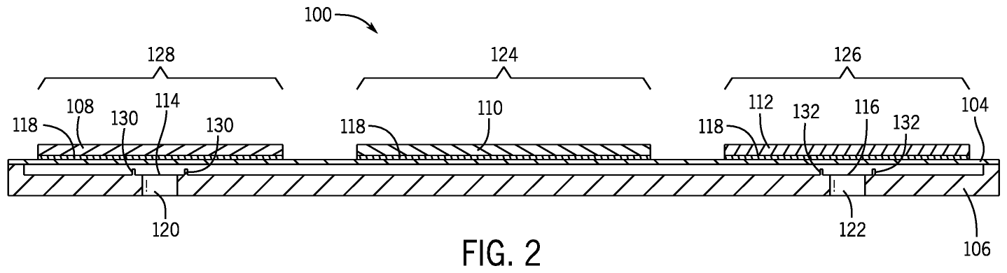

Micropump 100 is configured to maximize micropump efficiency per mm² (i.e., stroke volume per unit area per Watt). To this end, micropump 100 is an example cavity substrate that includes cavity 102 comprising three chambers 102a, 102b (outlet) and 102c (inlet) for fluid flow as shown in FIGS. 1 and 2. In this example, micropump 100 is a two-wafer structure including (1) silicon on insulator (SOI) wafer 104 (top wafer) that functions as a membrane for chambers 102a, 102b, 102c. SOI wafer 104 incorporates a buried oxide (BOX) layer and a silicon (Si) layer as known to those skilled in the art and (2) double sided polish (DSP) silicon wafer or layer 106. The handle silicon layer of the SOI wafer is removed to form the pump membrane. SOI wafer 104 sits between silicon wafer 106 and several piezoelectric actuators (transducers) 108, 110, 112 as shown and described below in more detail. (Micropump 100 is shown and described as a two-wafer structure as it provides several benefits including facilitating manufacturing process, but those skilled in the art know that it may be any number of wafers to achieve desired results.)

A metallization and conductive epoxy layer 118 binds piezoelectric actuators 108, 110 and 112 to SOI wafer 104 as known to those skilled in the art. In some detail, certain portions of layer 118 underneath corresponding piezoelectric actuators 108, 110, 112 act as ground electrodes while bonding pads 119 function as active electrodes as known to those skilled in the art. Wafer 106 includes inlet and outlet ports 114, 116 that communicate with chambers 102b and 102c of cavity 102 via channels 120, 122, respectively, that extend through the combined wafer structure (wafers 104, 106) as shown. (Note that wafers 106 may alternatively be SOI wafers as known to those skilled in the art.).

Micropump 100 includes pump section 124 and two valve sections 126, 128 that function together to pump fluid through cavity chambers 102a, 102b, 102c of micropump 100. Pump section 124 includes piezoelectric actuator 110 that is layered on top of silicon layer 104 (via metallization layer 118) and upon application of voltage, positive or negative, piezoelectric actuator 110 functions to pump or deform/bend silicon layer 104 to draw into or displace liquid contents into cavity chamber 102a from either port 114 or port 116 as desired. (Micropump operation is discussed in more detail below.)

Cavity chamber 102a is a pumping chamber that is considered part of or used by pump section 124. Valve sections 122, 124 include piezoelectric actuators 108, 112 respectively, as well as valve seats 130, 132, respectively. Valve seats 130, 132 are configured to extend into cavity chambers 102b, 102c and to define the introduction of channels 120, 122 from inlet/outlet ports 114, 116. Cavity chambers 102b, 102c are valve or valving chambers that are considered part of or used by valve sections 126, 128, respectively.

As described above, piezoelectric actuators 108, 112 are layered on top of SOI wafer 104 (via metallization layer 118). Piezoelectric actuators 108, 112 are configured to compress against SOI wafer 104 (membrane) to reach and seal valve seats 130, 132 to thereby discontinue flow through inlet/outlet ports 114, 116, respectively as needed for proper pump performance, as known to those skilled in the art.

In operation, the voltages to the piezoelectric actuators 108, 110, 112 can be controlled by a pump controller. This offers the ability to tailor any specific actuation sequence of each chamber to generate the necessary pressure changes to pump the insulin. At zero voltage for example, wafer 104 (membrane) remains flat, with no movement. When a positive voltage is applied, wafer 104 deforms upwards causing it to deflect upwards with it. When a negative voltage is applied, wafer 104 deforms downward causing it to deflect downward with it. In one example sequence, micropump 100 undergoes a four-phase sequence (scheme) to control the actuators 108, 110, 112.

The actuation scheme comprises of four phases a fluidic channel opening phase, pump fill phase, transfer phase, and delivery phase of the peristaltic micropump 100. Relatively fast actuation from the piezoelectric actuators are necessary to be able to achieve the right pressure changes across the three chambers in order for the insulin to be pumped through the micropump 100.

In the fluidic channel opening phase, wafer 104 over the valve section 128 is opened with a positive applied voltage allowing insulin or other medicament to be drawn into the valve chamber 102c from the reservoir. During this phase, the pump membrane (wafer 104) is deflected downward, and the valve section 126 is tightly closed (wafer 104 reaches valve seat 132 closing outlet port 116) in consequence of a negative applied voltage. At the end of this phase, the pressure within valve chamber 102c and pump chamber 102a is relaxed to the value of the inlet pressure.

The fill phase is initiated by the upwards deflection of the wafer 104 (valve section 128) as well as the upward deflection of the wafer (pump section 124) in consequence of a positive applied voltage creating negative pressure in the pump chamber 102a, enabling the insulin flow from the valve chamber 102c into the pump chamber 102a. During this phase, section 126 is deflected downward closing the valve seat 132 in consequence of a negative voltage applied. At the end of the fill phase, the initial negative pressure generated in the pump chamber 102a equilibrates to the inlet pressure.

The transfer phase is initiated by the simultaneous closing of the valve chamber 102c (wafer 104 closes off valve seat 130) and opening of the outlet port 116 or valve chamber 102b (wafer 104 closes off valve seat 132). The increased pressure generated from the inlet chamber and negative pressure generated from valve chamber 102b enables the insulin to travel through pump chamber 102a into valve chamber 102b. At the end of this phase, the pressure in pump chamber 102a and valve chamber 102b equilibrates to the outlet pressure.

Finally, in the delivery phase, the propelled volume is released from the micropump 100 as a consequence of the downward deflection of the wafer 104 (pump membrane), increasing the pressure both in pump chamber 102a and valve chamber 102b, causing the insulin to be delivered out of peristaltic micropump 100 through the outlet channel. At the end of this phase, the pressure is relaxed to the value of the valve chamber 102b.

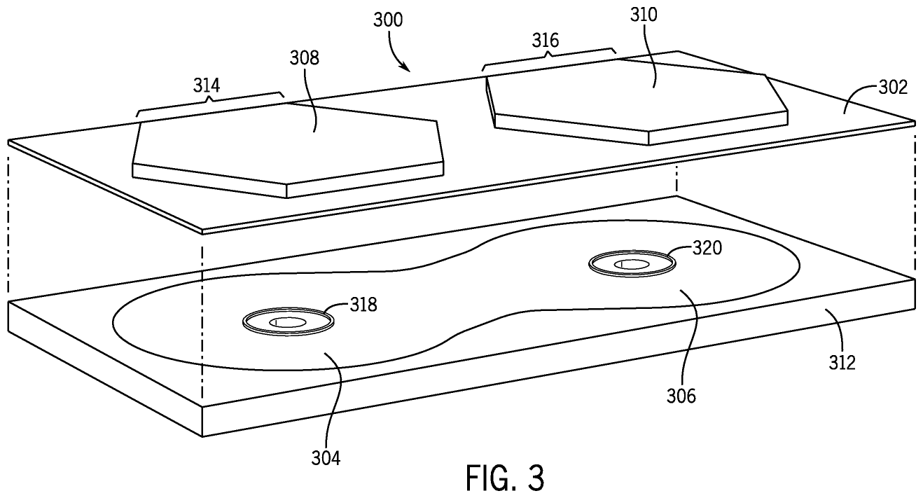

Structurally, note that a micropump may include any number of pumps and/or valves as described herein. That is, micropump may include any number of chambers such as (for example) one or two chambers as shown in FIG. 3 (and described in detail below) or four chambers, each chamber functioning as a valve or a pump or both in certain instances.

As described above and in the example shown in FIGS. 1 and 2, micropump 100 incorporates cavity 102 with three cavity chambers 102a, 102b, 102c that form a fluid path through micropump 100 from inlet port 114 to outlet port 116. In this example, the three chambers are configured as three circular (round) sections with connecting tapered channels (102d, 102e) therebetween, that form a dual oblong or dual overlapping hourglass configuration for cavity 102. Connecting the three chambers with channels with curved edges reduces potential for air pockets (bubbles). That is curved lines that create the channels help reduce bubble generation and accumulation. However, the channels and chambers may be configured to any size (e.g., square or rectangular chambers) to achieve desired results as known to those skilled in the art.

In this example, chamber 102a, i.e., its circular section, is larger in diameter than the diameters of circular sections of chambers 102b, 102c, for example to optimize compression ratio within chamber 102a. The difference in diameters may be determined to help optimize compression ratio within chamber 102a as well as enable the micropump to self prime. The diameter ratio sizing is set to maximize pressure generated/required to move fluid through chamber 102a over the total amount of fluid within chamber 102a. In other examples, all three chambers may be the same size in diameter or have multiple size diameters. For example, the (outlet) valve chamber 102b may be configured to be smaller than (inlet) valve chamber 102c itself to improve low compression ratio, increase hydraulic resistance and reduce leakage.

The width of a connecting channel 102d, 102e between circular sections of chambers 102a, 102b, 102c as described above is maximized to avoid or prevent membrane sectional cross talk or interference between neighboring circular sections during operation as well as avoid generating air pockets (bubbles) as known to those skilled in the art. The width of the connecting channels of the three chambers may be for example between 0.25 mm and 2 mm. However, the width may be any measurement to achieve desired results as known to those skilled in the art.

The chamber depth is optimized to avoid creating dead volume or dampening. That is, chamber depth is design to prevent the membrane from reaching the bottom of the chamber during operation. In this example, the chamber depth of all three chambers is held at a consistent value to reduce overall hydraulic resistance within cavity 102. The chamber(s) depth may be 15-40 um to avoid hydraulic resistance but the depth may have any other measurement to achieve desired results as known to those skilled in the art. Inlet and outlet ports 114, 116 for example may have lengths between 250-600 um and diameters between 100-1000 um to help adjust hydraulic resistance within cavity 102, i.e., to manipulate velocity of fluid to control bubble generation and turbulent flow within cavity 102. However, the inlet and outlet ports 114, 116 may be of any length to achieve desired results. The downward deflection of pump membrane 104 to chamber depth may be for example between 50-90% to help maximize the compression ratio while also avoiding dampening due to squeeze film effects.

SOI wafer 104, as described above, is a membrane that is configured to maximize deflection while at the same achieving enough stiffness to reduce the impact of back pressure within cavity 102 of micropump 100. To this effect, the membrane is preferably configured to have a silicon thickness to silicon membrane diameter ratio of 0.75-2%. Piezoelectric actuators 108, 112 to silicon membrane diameter are configured to be optimized for valve sections 126, 128 to maximize membrane deflection and pump section 124 to maximize stroke volume of membrane 104 as known to those skilled in the art. The piezoelectric actuator thickness to silicon membrane thickness may be for example sized 1-1.8× to optimize for flow rate or back pressure. The ratio may be other valves to achieve desired results as known to those skilled in the art. The piezoelectric diameter to thickness may be for example between 45-70× to help optimize membrane deflection and stiffness with regard to applicable voltage and changes electric field.

In FIG. 3, micropump 300 is also a two-wafer structure including (1) silicon on insulator (SOI) wafer 302 (top wafer) that functions as a membrane for chambers 304, 306. SOI wafer 302 sits between piezoelectric actuators (transducers) 308, 310 and silicon wafer or layer 312 as shown. Micropump 300 includes two section 314, 316 that incorporate piezoelectric actuators 308, 310, chambers 304, 306 and valve seats 318, 320 respectively. Chambers 304, 306 may be configured as a pump and valve, two pumps or two valves.

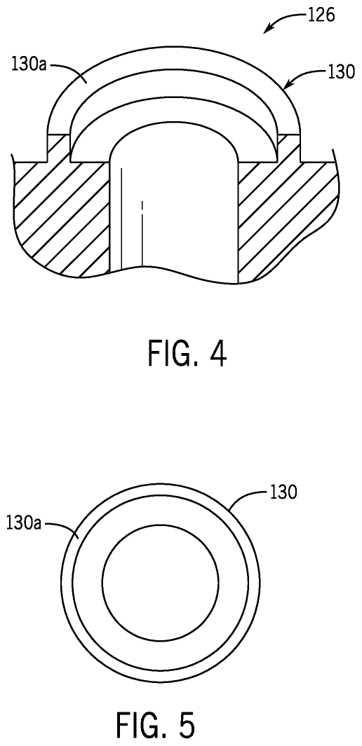

FIGS. 4 and 5 depicts a cross-sectional perspective view and top view of valve section 126 of the micropump 100 in FIG. 1 including valve lip 130a of valve seat 130. Valve section 130 is similar to valve seat 132 in the example shown.) Changing the radius or diameter of valve lip 130a (either lip) changes the hydraulic resistance. Decreasing the diameter increases the hydraulic resistance which decreases leakage. Reducing valve lip width, i.e., thickness helps reduce stiction (membrane sticking to valve lip). The diameter of outlet port 116 may be made smaller than diameter of inlet port 114 to reduce outlet flow. This reduces free flow through input port and ultimately reduces flow through the chambers.

With respect to valve sections 126, 128, a gap height itself between valve seats 130, 132 and SOI wafer membrane 104 is configured (e.g., sized) to create hydraulic resistance and prevent free flow. The gap may be configured to be between 0-15 um for example to improve self-priming ability, reduce hydraulic resistance, increase bubble tolerance and free flow prevention. Valve gap should be sufficient to enable closure of the valve seat.

The width layer 104 is configured to balance thickness and stiffness for optimal ratio of membrane to piezoelectric actuator thickness. That is, the membrane width must be configured to actuate and move up and down to close valve lips but large/stiff enough to generate suction in chamber to draw or displace fluid contents as described above. For example, the membrane thickness may be between 30-70 percent of piezoelectric actuator thickness to obtain the benefit of both stiffness and deflection. This is described again below.

In addition, the membrane is also configured to optimize the valve gap height during actuation while reducing hydraulic resistance. For example, the width of relative membrane may be 400-1000 um to optimize the valve gap height during actuation while reducing hydraulic resistance. Valve lip 130a width may be 8-100 um to provide mechanical stability and adequate sealing of valve lips. The ratio of actuated the gap height to lip width is sized to help for self-priming achievement due to the Young Laplace pressure drop.

Valve sections 126, 128 in FIGS. 1-2 are configured as active valves whereby piezoelectric actuators 108, 112 are actuated (force) to shut off valve, i.e., cause wafer membrane 104 to deflect and cover the valve seats. This decreases leakage and improves sealing in the channels 120, 122.

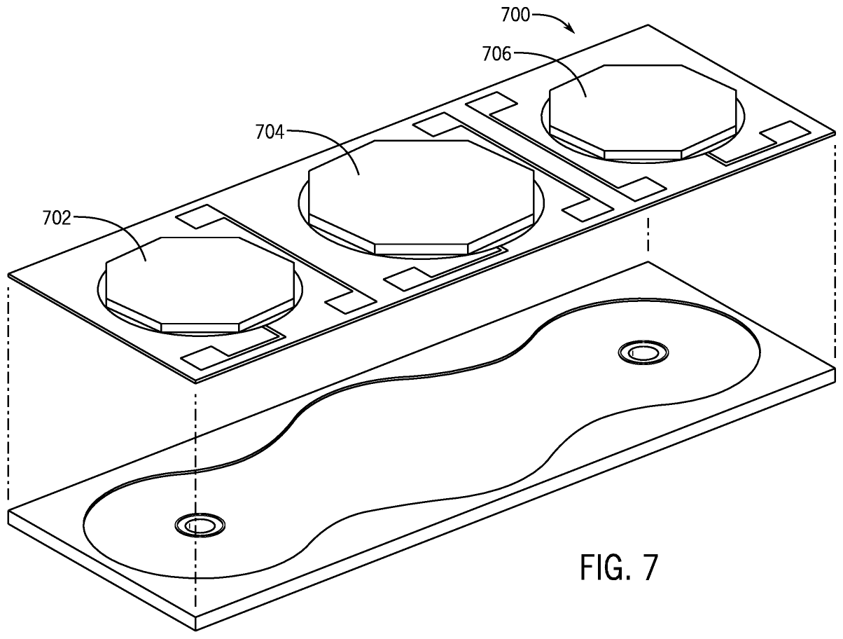

Piezoelectric actuators 108, 110, 112 are hexagonal or octagonal shape but may be any shape as desired. As shown in FIG. 7 and described below, micropump 700 may incorporate piezoelectric actuators 702, 704, 706 that are octagonal shape which increases volumetric deflection over the area of the membrane. This improves volumetric deflection and therefore pumping and self-priming.

The measurement values described hereinabove for various micropump 100 dimensions etc. are example values. Those skilled in the art know that such values may differ to compensate for other values or to achieve desired results.

FIG. 6 depicts a block diagram of example components of device 600 for delivering insulin or other medicament of an infusion system as described in detail above. Specifically, device 600 incorporates several components or modules (not shown) in the fluidic pathway including reservoir 602 for storing the insulin or other medicament, micropump 604 for pumping the insulin or other medicament as described above, sensors 606 (e.g., pressure) for sensing various parameters in the system and user and tubing connecting infusion catheter or needle 608 to reservoir 602. Device 600 also includes microcontroller unit (MCU) 610 and battery and power controller 612, CGM sensor 616 and infusion catheter or needle 608. CGM is an example sensor for sensing a biomarker. CGM sensor, as known to those skilled in the art, tracks user glucose levels and permits those levels to be used in algorithms that control flow rate. MCU 610 controls the operation of micropump 604.

Reservoir 602 is configured to receive and store insulin or other medicament for its delivery over a course of about three days, or as needed. However, reservoir size may be configured for storing any quantity of fluid as required.

MCU 610 electronically communicates with sensors 606 and micropump 600 as well as the CGM sensor 616, as the monitoring components. Among several functions, MCU 610 operates to control the operation of micropump 504 to deliver insulin or other medicament through infusion catheter/needle 608 from reservoir 602 at specific doses, i.e., flow rates over specified time intervals, based on CGM data (or other sensed data) converted to desired flow rate via control algorithms.

Battery and power controller 612 controls the power to MCU 610 and micropump 602 to enable those components to function properly as known to those skilled in the art. CGM sensor 616 is powered by battery and power controller 612 through MCU 610.

The components of device 600 depict only a few components. Those skilled in the art know that device 600 including additional components.

In addition, as mentioned above, device 600 may also be used to deliver other medicaments (i.e., other than insulin) and sense other biomarkers (i.e., other than glucose). Examples of medicaments or medication include small molecule pharmaceutical solutions, large molecule or protein drug solutions, saline solutions, blood or other fluids known to those skilled in the art.

FIG. 7 depicts a perspective exploded view of an example micropump 700 for a device for delivering insulin to a user. Micropump 700 is similar in structure and function as micropump 100 except that piezoelectric actuators 702, 704, 706 in FIG. 7 are octagonal shaped (not hexagonal shaped as in FIG. 1.). Piezoelectric actuators 702, 704, 706 have increased volumetric deflection across the surface area because the octagonal shape covers a greater are of the membrane.

It is to be understood that the disclosure teaches examples of the illustrative embodiments and that many variations of the invention can easily be devised by those skilled in the art after reading this disclosure and that the scope of the present invention is to be determined by the claims below.

What is claimed is:

1. A MEMS device for a device for delivering medicament into a subcutaneous tissue of a user, the device for delivering medicament configured to be mounted to the user, the MEMS device including an inlet port and outlet port, the MEMS device configured as a micropump to pump the medicament from the inlet port to the outlet port, the MEMS device comprising:
first and second wafers that define a cavity therebetween that communicates with the inlet and outlet ports, thereby creating a fluid path for a flow of the medicament from the inlet port to the outlet port, the first wafer configured as a membrane, the cavity comprises:
a first chamber configured as a pumping chamber of the micropump; and
a second chamber and a third chamber configured as valve chambers of the micropump, wherein the second chamber communicates with the first chamber and the inlet port and the third chamber communicates with the first chamber and the outlet port and wherein the first, second chamber and third chamber form the fluid path through the micropump from the inlet port to the outlet port, wherein the first wafer as a membrane is configured to deflect creating a pressure difference within the cavity and thereby draw medicament from the inlet port into the first chamber or displace medicament from the first chamber toward the outlet port.

2. The MEMS device of claim 1 wherein the first, second and third chambers are configured as first, second and third circular sections with connecting channels between the first and second chambers and the first and third chambers.

3. The MEMS device of claim 2 wherein the connecting channels are tapered and comprise curved edges to prevent bubble generation within the fluid path.

4. The MEMS device of claim 2 wherein the first chamber has a diameter that is greater than diameters of the second and third chambers.

5. The MEMS device of claim 2 wherein the first, second and third chambers have equal diameters.

6. The MEMS device of claim 1 further comprising (1) a pump section including a piezoelectric actuator layered on the first wafer over the first chamber, the piezoelectric actuator configured to deform the first wafer to draw into or displace medicament from the first chamber and (2) first and second valve sections, each including a piezoelectric actuator layered on the first wafer over the second and third chambers.

7. The MEMS device of claim 6 wherein the first and second valve sections further include first and second valve seats, respectively that extend into the second and third chambers, respectively, the first and second valve seats define an introduction to first and second channels from the inlet and outlet ports, respectfully, the first piezoelectric actuators of the first and second valve sections configured to compress the first wafer to reach and seal the first and second valve seats to thereby discontinue flow through the inlet and outlet ports.

8. The MEMS device of claim 6 wherein the first wafer and the first, second and third chambers are configured to maximize stroke volume while overcoming back pressure through the micropump.

9. The MEMS device of claim 1 wherein the second chamber has a diameter larger than the first chamber and third chamber and the first chamber has a diameter larger than the third diameter.

10. The MEMS device of claim 1 wherein the medicament is insulin.

11. A device for delivering insulin to a user for diabetes management, the device configured to be mounted the user, the device comprising:
an infusion catheter for infusing the insulin into a subcutaneous tissue of the user;
a MEMS device configured as a micropump for pumping the insulin through the micropump, the MEMS device in fluid communication with the infusion catheter, the MEMS device comprising:
an inlet port for receiving the insulin and outlet port for releasing the insulin to supply;
first and second wafers that define a cavity that communicates with the inlet and outlet ports, thereby creating a fluid path for a flow of the insulin from the inlet port to the outlet port, the cavity including a first chamber configured as a pump chamber and/or a valve chamber of the micropump and a second chamber configured as a pump chamber and/or a valve chamber, the first chamber and second chamber in communication therewith creating the fluid path between the inlet and outlet ports;
a first piezoelectric actuator layered on the first wafer and configured to deform the first wafer relative to the first chamber;
a second piezoelectric actuator layered on the first wafer and configured to deform the first wafer relative to the second chamber,
wherein the first wafer as a membrane is configured, upon deformation, to create a pressure difference within the cavity and thereby draw insulin from the inlet port into the first chamber and/or second chamber or displace fluid from the first chamber and/or second chamber toward the outlet port.

12. The device of claim 11 wherein the cavity of the MEMS device further comprising a third chamber configured as a pump chamber of the micropump and a third piezoelectric actuator layered on the first wafer and configured to deform the first wafer relative to the third chamber.

13. The device of claim 12 wherein the first, second and third second chambers and first, second and third piezoelectric actuators are configured to maximize stroke volume while overcoming back pressure through the micropump.

14. The device of claim 11 further comprising a CGM sensor for measuring glucose level in the user.

15. The MEMS device of claim 12 wherein the first, second and third chambers are configured as first, second and third circular sections with connecting channels between the first and second chambers and the first and third chambers.

16. The MEMS device of claim 15 wherein the connecting channels are tapered and comprise curved edges to prevent bubble generation within the fluid path.

17. The MEMS device of claim 12 wherein the first chamber has a diameter that is greater than diameters of the second and third chambers.

18. The MEMS device of claim 11 wherein the first and second chambers have equal diameters.

19. A device for delivering insulin to a user for diabetes management, the device configured to be mounted the user, the device comprising:
an infusion catheter for infusing the insulin into a subcutaneous tissue of the user;
a MEMS device configured as a micropump for pumping the insulin through the micropump, the MEMS device in fluid communication with the infusion catheter, the MEMS device comprising:
an inlet port for receiving the insulin and outlet port for releasing the insulin to supply;
first and second wafers that define a cavity that communicates with the inlet and outlet ports, thereby creating a fluid path for a flow of the insulin from the inlet port to the outlet port, the cavity including a first chamber configured as a pump chamber, a second chamber configured as a valve chamber and a third chamber configured as a valve chamber, the first chamber in communication with the second chamber and third chamber therewith creating the fluid path between the inlet and outlet ports;
a first piezoelectric actuator layered on the first wafer and configured to deform the first wafer relative to the first chamber;
a second piezoelectric actuator layered on the first wafer and configured to deform the first wafer relative to the second chamber; and
a third piezoelectric actuator layered on the first wafer and configured to deform the first wafer relative to the third chamber,
wherein the first wafer as a membrane is configured, upon deformation, to create a pressure difference within the cavity and thereby draw insulin from the inlet port into the second chamber or displace insulin from the first chamber toward the outlet port.

20. The device of claim 19 wherein the first, second and third second chambers and first, second and third piezoelectric actuators are configured to maximize stroke volume while overcoming back pressure through the micropump.

21. The device of claim 19 further comprising a CGM sensor for measuring glucose level in the user.

22. The MEMS device of claim 19 wherein the first, second and third chambers are configured as first, second and third circular sections with connecting channels between the first and second chambers and the first and third chambers, respectively.

23. The MEMS device of claim 22 wherein the connecting channels are tapered and comprise curved edges to prevent bubble generation within the fluid path.

24. The MEMS device of claim 22 wherein the first chamber has a diameter that is greater than diameters of the second and third chambers.

25. The MEMS device of claim 22 wherein the first and second chambers have equal diameters.

26. A device for delivering medicament to a user, the device configured to be mounted the user, the device comprising:
a sensor for sensing a biomarker in the user;
an infusion catheter for infusing the medicament into tissue of the user based on the biomarker sensed by the sensor;
a MEMS device configured as a micropump for pumping the medicament through the micropump, the MEMS device in fluid communication with the infusion catheter, the MEMS device comprising:
an inlet port for receiving the medicament and outlet port for releasing the medicament to supply;
first and second wafers that define a cavity that communicates with the inlet and outlet ports, thereby creating a fluid path for a flow of the medicament from the inlet port to the outlet port, the cavity including a first chamber configured as a pump chamber, a second chamber configured as a valve chamber and a third chamber configured as a valve chamber, the first chamber in communication with the second chamber and third chamber therewith creating the fluid path between the inlet and outlet ports;
a first piezoelectric actuator layered on the first wafer and configured to deform the first wafer relative to the first chamber;
a second piezoelectric actuator layered on the first wafer and configured to deform the first wafer relative to the second chamber; and
a third piezoelectric actuator layered on the first wafer and configured to deform the first wafer relative to the third chamber,
wherein the first wafer as a membrane is configured, upon deformation, to create a pressure difference within the cavity and thereby draw medicament from the inlet port into the second chamber or displace medicament from the first chamber toward the outlet port.

27. The device of claim 26 wherein the medicament is insulin and the biomarker is glucose level.

28. A device for delivering insulin to a user for diabetes management, the device configured to be mounted the user, the device comprising:
an infusion catheter for infusing the insulin into a subcutaneous tissue of the user;
a MEMS device configured as a micropump for pumping the insulin through the micropump, the MEMS device in fluid communication with the infusion catheter, the MEMS device comprising:
an inlet port for receiving the insulin and outlet port for releasing the insulin to supply;
first and second wafers that define a cavity that communicates with the inlet and outlet ports, thereby creating a fluid path for a flow of the insulin from the inlet port to the outlet port, the cavity including a first chamber configured as a pump chamber and as a circular section, a second chamber configured as a valve chamber and as a circular section, a third chamber configured as a valve chamber and as a circular section, a first channel between the first chamber and second chamber and a second channel between the first chamber and third chamber, the first and second channels thereby enabling communication within the cavity and the flow of insulin between the inlet and outlet ports;
a first piezoelectric actuator layered on the first wafer and configured to deform the first wafer relative to the first chamber;
a second piezoelectric actuator layered on the first wafer and configured to deform the first wafer relative to the second chamber; and
a third piezoelectric actuator layered on the first wafer and configured to deform the first wafer relative to the third chamber,
wherein the first wafer as a membrane is configured, upon deformation, to create a pressure difference within the cavity and thereby draw insulin from the inlet port into the second chamber or displace insulin from the first chamber toward the outlet port.

29. The device of claim 28 wherein the first and second channels have curved edges.

30. The device of claim 28 wherein the first and second channels are tapered and comprise curved edges.

31. The device of claim 28 wherein at least one of the first, second and third piezoelectric actuators is polygonal shaped to maximize volumetric deflection in first, second and third chambers respectively.

32. The device of claim 28 wherein at least one of the first, second and third piezoelectric actuators is octagonal shaped to maximize volumetric deflection in first, second and third chambers respectively.

33. The device of claim 28 further comprising a CGM sensor for measuring glucose level in the user.
